(19) United States
(12) Patent Application Publication
(10) Pub. No.: US 2007/0197529 A1
(43) Pub. Date: Aug. 23, 2007

(54) Isolated desfluoro-linezolid, preparation thereof and its use as a reference marker and standard

(76) Inventors: Viviana Braude, Kadima (IL); Nina Finkelstein, Herzliya (IL)

Correspondence Address: KENYON & KENYON LLP, ONE BROADWAY, NEW YORK, NY, 10004, US

(21) Appl. No.: 11/607,423

(22) Filed: Nov. 30, 2006

Related U.S. Application Data
(60) Provisional application No. 60/742,026, filed on Dec. 1, 2005.

(51) Int. Cl. A61K 31/5377; C07D 413/02

(52) U.S. Cl. 514/235.2; 544/139

(57) ABSTRACT

The present invention provides an isolated linezolid impurity, desfluoro linezolid, the preparation thereof and its use as a reference standard.

### CROSS REFERENCE TO RELATED APPLICATIONS

The present application claims the benefit of the following U.S. Provisional Patent Application No. 60/742,026, filed Dec. 1, 2005. The contents of this application is incorporated herein by reference.

### FIELD OF THE INVENTION

The present invention relates to isolated desfluoro-linezolid, methods for the preparation and detection thereof, and methods of using desfluoro-linezolid as a reference marker.

### BACKGROUND OF THE INVENTION

Linezolid [(S)-N-[[3-(3-Fluoro-4-morpholinyl)phenyl]-2-oxo-5-oxazolidinyl]methyl]acetamide] is an antimicrobial agent. Linezolid is an oxazolidinone, having the empirical formula C₁₆H₂₀FN₃O₄ and the following structure:

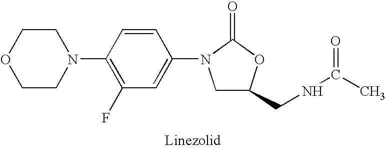

Linezolid is described in The Merck Index (13th edition, Monograph number: 05526, CAS Registry Number: 165800-03-3) as white crystals, with a melting point of 181.5-182.5° C. Linezolid, as well as a process for its preparation, is described in U.S. Pat. No. 5,688,792 (Example 5), European Patent No. 717738, Israeli Patent No. 110,802, Canadian Patent No. 2,168,560, and International Patent Publication WO 95/07271.

Linezolid is marketed in the United States by Pfizer, Inc. as an injection, as tablets, and as an oral suspension under the name ZYVOX®. Its main indications are nosocomial pneumonia, skin and skin-structure infections, and vancomycin-resistant Enterococcus faecium infections.

U.S. Pat. No. 5,688,792 describes linezolid and its use for the treatment of microbial infections. This patent also describes the following method for the preparation of linezolid:

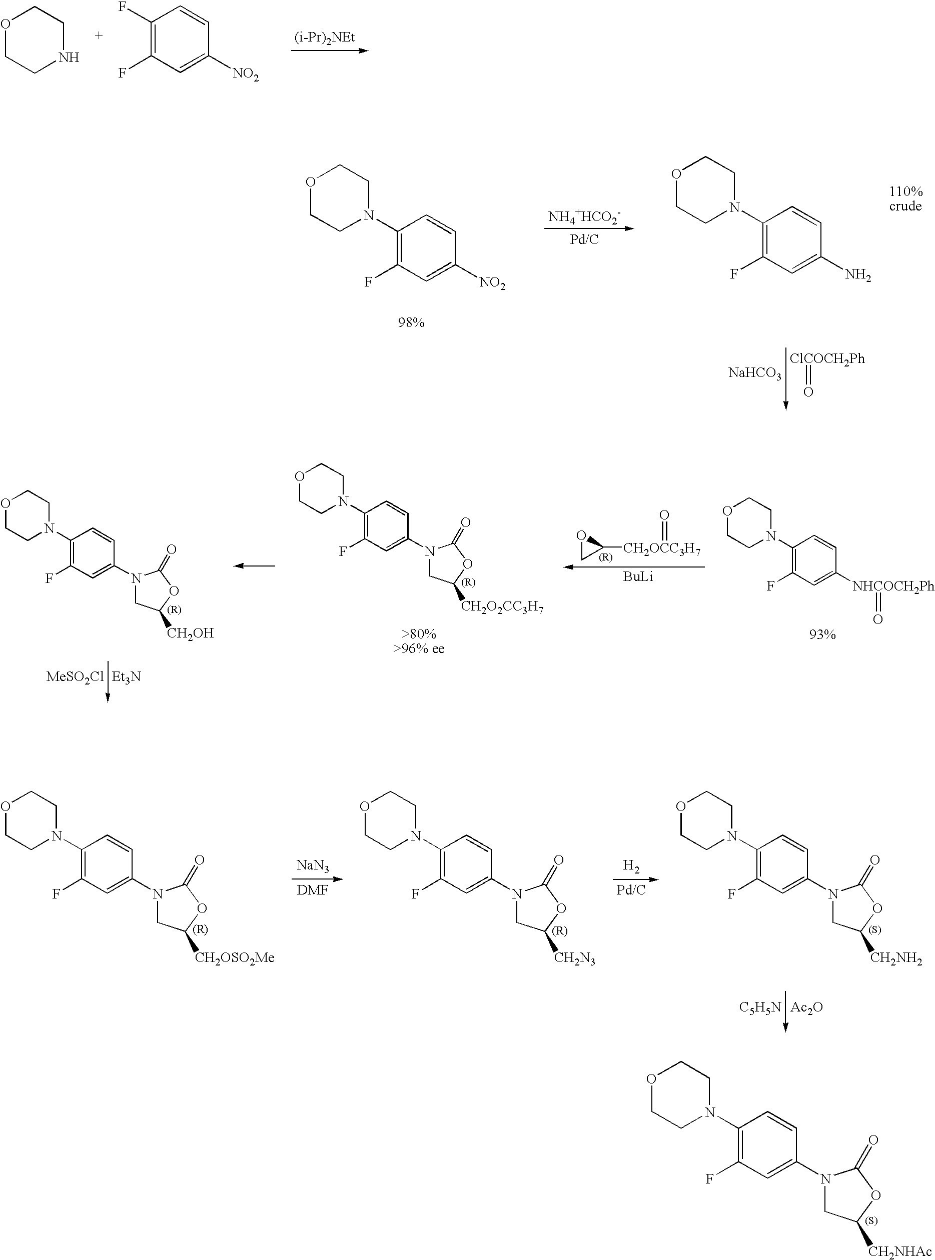

This method of preparation was also described in Bricker, et al., J. Med. Chem., 39, 673-679 (1996), where it was stated that the above route avoids the use of phosgene to make the carbamate precursor of the oxazolidinone ring. The authors also disclose that the use of NaN₃ can be avoided by using potassium phthalimide, followed by deblocking of the phthalimide with aqueous methyl amine.

An analysis of the commercial tablet ZYVOX® shows the presence of desfluoro linezolid as an impurity of linezolid. An HPLC chromatogram of ZYVOX® is depicted in FIG. 1. The desfluoro linezolid haviong a relative retention time (RRT) of 0.69 compared to the retention time of linezolid.

It is well known in the art that, for human administration, safety considerations require the establishment, by national and international regulatory authorities, of very low limits for identified, but toxicologically uncharacterized impurities, before an active pharmaceutical ingredient (API) product is commercialized. Typically, these limits are less than about 0.15 percent by weight of each impurity. Limits for unidentified and/or uncharacterized impurities are obviously lower, typically, less than 0.1 percent by weight. Therefore, in the manufacture of APIs, purity of the products, such as linezolid, is required before commercialization, as well as purity of the active agent in the manufacture of formulated pharmaceuticals.

It is also known in the art that impurities in an API may arise from degradation of the API itself, which is related to the stability of the pure API during storage, and the manufacturing process, including the chemical synthesis. Process impurities include unreacted starting materials, chemical derivatives of impurities contained in starting materials, synthetic byproducts, and degradation products.

In addition to stability, which is a factor with respect to the shelf life of the API, the purity of the API produced in the commercial manufacturing process is clearly a necessary condition for commercialization. Impurities introduced during commercial manufacturing processes must be limited to very small amounts, and are preferably substantially absent. For example, the ICH Q7A guidance for API manufacturers requires that process impurities be maintained below set limits by specifying the quality of raw materials, controlling process parameters, such as temperature, pressure, time, and stoichiometric ratios, and including purification steps, such as crystallization, distillation, and liquid-liquid extraction, in the manufacturing process.

The product mixture of a reaction is rarely a single compound with sufficient purity to comply with pharmaceutical standards. Side products and byproducts of the reaction and adjunct reagents used in the reaction will, in most cases, also be present in the product mixture. At certain stages during processing of an API, such as linezolid, it must be analyzed for purity, typically, by HPLC or GC analysis, to determine if it is suitable for continued processing and, ultimately, for use in a pharmaceutical product. The API need not be absolutely pure, as absolute purity is a theoretical ideal that is typically unattainable. Rather, purity standards are set with the intention of ensuring that an API is as free of impurities as possible, and, thus, are as safe as possible for clinical use. As discussed above, in the United States, the Food and Drug Administration guidelines recommend that the amounts of some impurities be limited to less than 0.1 percent by weight.

Generally, side products, byproducts, and adjunct reagents (collectively “impurities”) are identified spectroscopically and/or with another physical method, and then associated with a peak position, such as that in a chromatogram, or a spot on a TLC plate. (Strobel p. 953, Strobel, H. A.; Heineman, W. R., Chemical Instrumentation: A Systematic Approach, 3rd ed. (Wiley & Sons: New York 1989)). Thereafter, the impurity can be identified, e.g., by its relative position in the chromatogram, where the position in a chromatogram is conventionally measured in minutes between injection of the sample on the column and elution of the particular component through the detector. The relative position in the chromatogram is known as the “retention time.” The retention time varies daily, or even over the course of a day, based upon the condition of the instrumentation, as well as many other factors. To mitigate the effects such variations have upon accurate identification of an impurity, practitioners use the “relative retention time” (“RRT”) to identify impurities. (Strobel p. 922). The RRT of an impurity is its retention time divided by the retention time of a reference marker. In theory, linezolid itself could be used as the reference marker, but as a practical matter it is present in such a large proportion in the mixture that it can saturate the column, leading to irreproducible retention times, as the maximum of the peak can wander (Strobel, FIG. 24.8 (b), p. 879, illustrates an asymmetric peak observed when a column is overloaded). Thus, it may be advantageous to select a compound other than the API that is added to, or present in, the mixture in an amount sufficiently large to be detectable and sufficiently low as not to saturate the column, and to use that compound as the reference marker.

Those skilled in the art of drug manufacturing research and development understand that a compound in a relatively pure state can be used as a “reference standard.” A reference standard is similar to a reference marker, which is used for qualitative analysis only, but is used to quantify the amount of the compound of the reference standard in an unknown mixture, as well. A reference standard is an “external standard,” when a solution of a known concentration of the reference standard and an unknown mixture are analyzed using the same technique. (Strobel p. 924, Snyder p. 549, Snyder, L. R.; Kirkland, J. J. Introduction to Modern Liquid Chromatography, 2nd ed. (John Wiley & Sons: New York 1979)). The amount of the compound in the mixture can be determined by comparing the magnitude of the detector response. See also U.S. Pat. No. 6,333,198, incorporated herein by reference.

The reference standard can also be used to quantify the amount of another compound in the mixture if a “response factor,” which compensates for differences in the sensitivity of the detector to the two compounds, has been predetermined. (Strobel p. 894). For this purpose, the reference standard is added directly to the mixture, and is known as an “internal standard.” (Strobel p. 925, Snyder p. 552).

The reference standard can even be used as an internal standard when, without the addition of the reference standard, an unknown mixture contains a detectable amount of the reference standard compound using a technique known as “standard addition.” In a “standard addition,” at least two samples are prepared by adding known and differing amounts of the internal standard. (Strobel pp. 391-393, Snyder pp. 571, 572). The proportion of the detector response due to the reference standard present in the mixture without the addition can be determined by plotting the detector response against the amount of the reference standard added to each of the samples, and extrapolating the plot to zero. (See, e.g., Strobel, FIG. 11.4 p. 392).

There is a need to isolate the desfluoro linezolid impurity. This impurity may also be used as a reference marker and/or standard.

### SUMMARY OF THE INVENTION

In one embodiment, the invention is directed to isolated desfluoro linezolid, of the following structure: 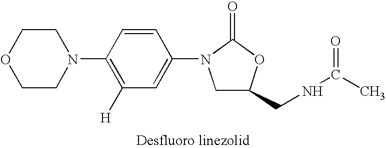
as well as the preparation thereof.

In yet another embodiment, the invention is directed to a method of using desfluoro linezolid as a reference marker to analyze the purity of linezolid.

In yet another embodiment, the invention is directed to a method of using desfluoro linezolid as a reference standard to quantify the amount of a desfluoro linezolid impurity in a sample of linezolid.

In a further embodiment, the invention is directed to analytical methods for testing and determining the impurity profile of linezolid. These methods are also suitable for analyzing and assaying linezolid and desfluoro linezolid.

### BRIEF DESCRIPTION OF THE FIGURES

FIG. 1 shows the HPLC analysis of the commercial tablet ZYVOX®

FIG. 2 shows the ¹H-NMR spectra of desfluoro linezolid

FIG. 3 shows the ¹³C-NMR spectra of desfluoro linezolid

FIG. 4 shows the IR spectra of desfluoro linezolid

FIG. 5 shows the mass spectra of desfluoro linezolid

FIG. 6 shows the HPLC analysis of linezolid.

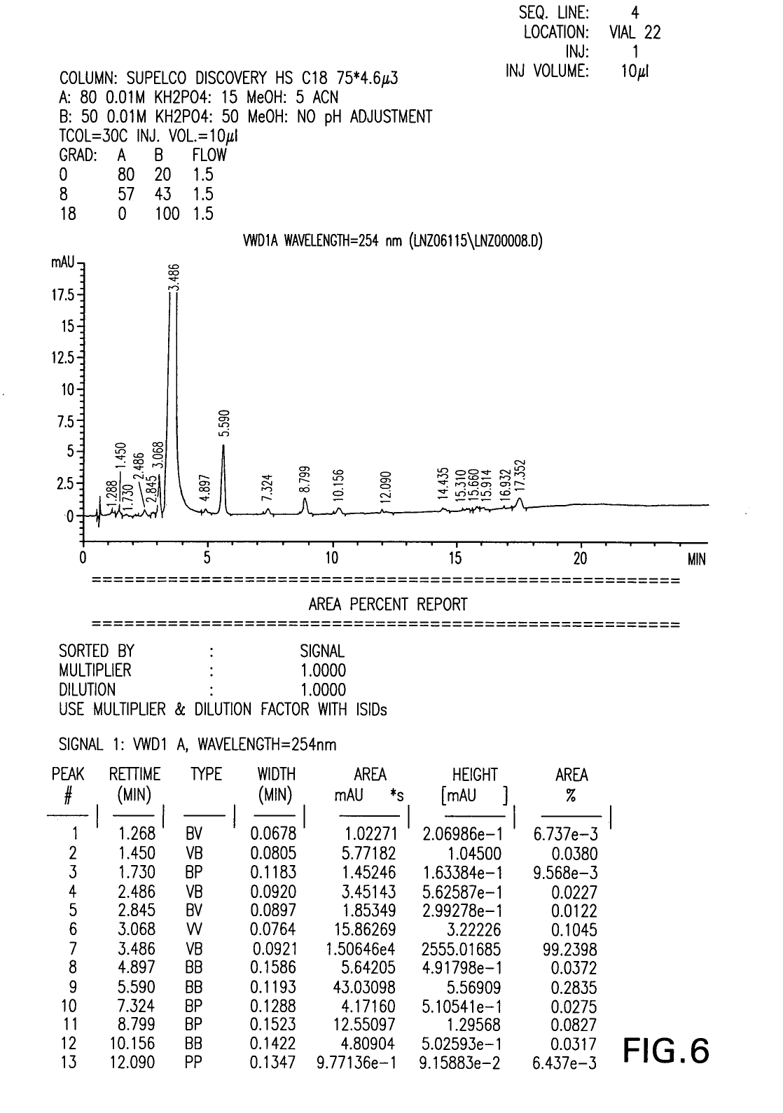

### DETAILED DESCRIPTION OF THE INVENTION

As used herein, the term “reference standard” refers to a compound that may be used both for quantitative and qualitative analysis of an active pharmaceutical ingredient. For example, the HPLC retention time of the reference standard compound allows a relative retention time with respect to the active pharmaceutical ingredient to be determined, thus making qualitative analysis possible. Furthermore, the concentration of the compound in solution before injection into an HPLC column allows the areas under the HPLC peaks to be compared, thus making quantitative analysis possible.

A “reference marker” is used in qualitative analysis to identify components of a mixture based upon their position, e.g., in a chromatogram or on a Thin Layer Chromatography (TLC) plate (Strobel pp. 921, 922, 953). For this purpose, the compound does not necessarily have to be added to the mixture if it is present in the mixture. A “reference marker” is used only for qualitative analysis, while a reference standard may be used for quantitative or qualitative analysis, or both. Hence, a reference marker is a subset of a reference standard, and is included within the definition of a reference standard.

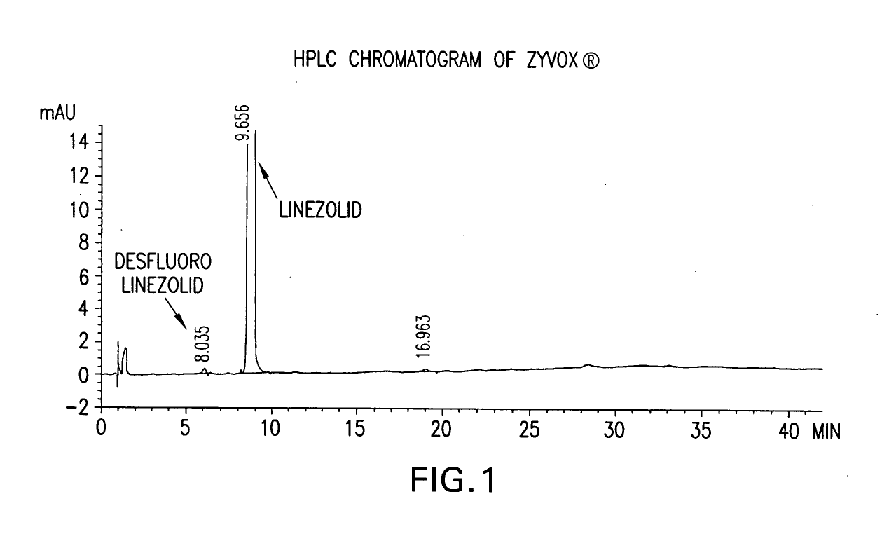

The present invention provides isolated desfluoro linezolid of the following structure: 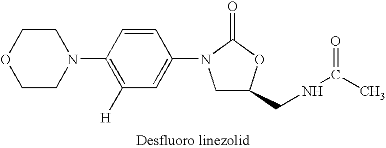
As illustrated in FIG. 1, this impurity is ideal for use as a reference standard since it is detectable by HPLC, and yet it is present in much less amounts than linezolid, having a RRT of 0.69 compared to the retention time of linezolid.

The isolated desfluoro linezolid is pure. Preferably it has about 95% purity by weight with respect to other compounds, including linezolid. Preferably, the desfluoro linezolid is isolated in about 99.3% purity by weight. Thus, the isolated desfluoro linezolid contains less than about 5%, preferably less than about 2%, and even more preferably less than about 1%, by weight, linezolid.

The isolated desfluoro linezolid of the present invention can be characterized by data selected from: ¹H NMR (400 MHz, DMSO-d6) δ (ppm): 1.83 (s), 3.04 (brt), 3.40 (t), 3.68 (m), 3.72 (brt), 4.04 (t), 4.67 (m), 6.95 (d), 6.95 (d), 7.37 (d), 7.37 (d) and 8.21 (t); ¹³C NMR (100 MHz, DMSO-d6) δ (ppm): 22.8, 41.9, 48.0, 49.2, 66.5, 71.7, 115.9, 115.9, 119.9, 119.9, 130.9, 148.0, 154.7, 170.0; EI+m/z (MH⁺): 319; and IR spectra on KBr at 1523, 1555, 1656, 1731, 2830, 2926, 2968 and 3311 cm⁻¹.

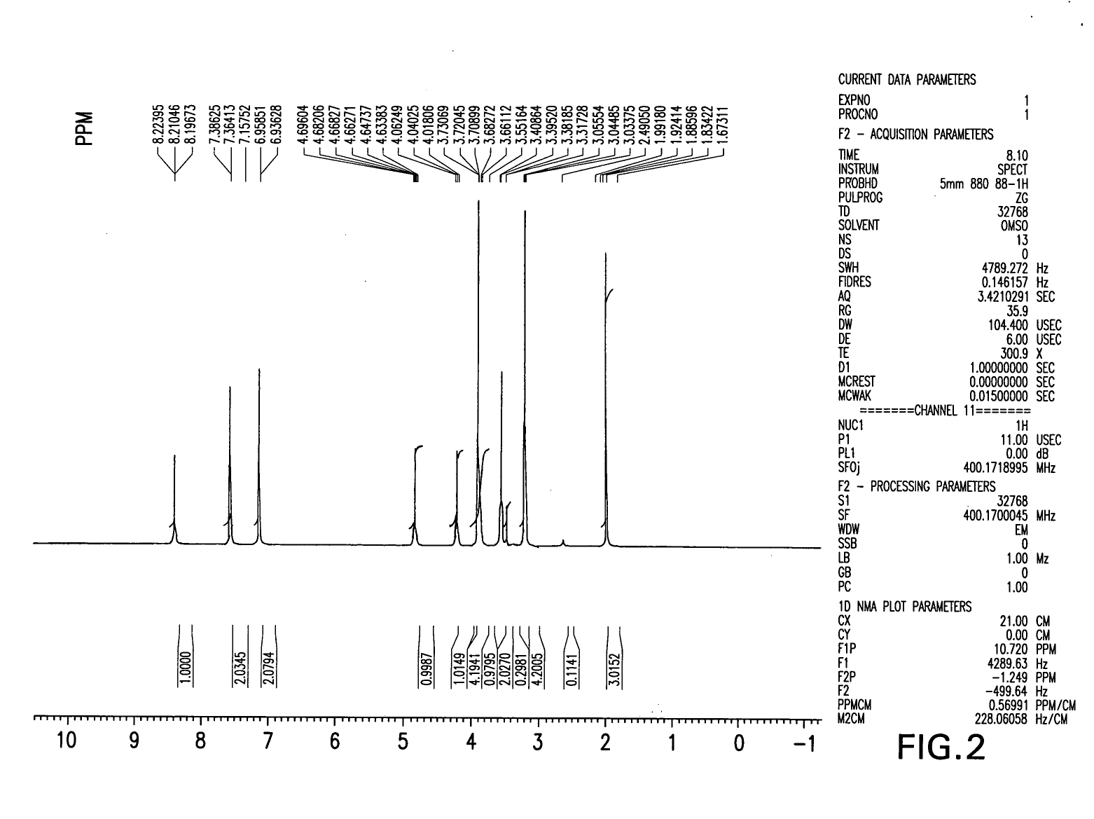

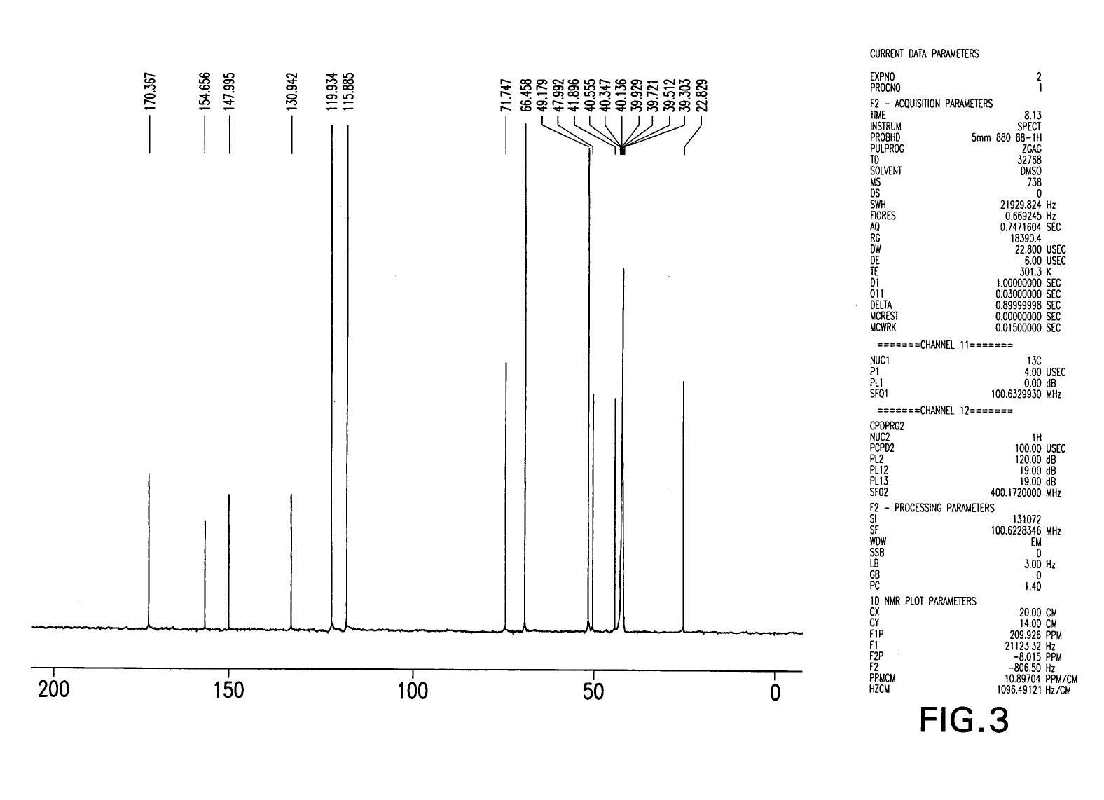

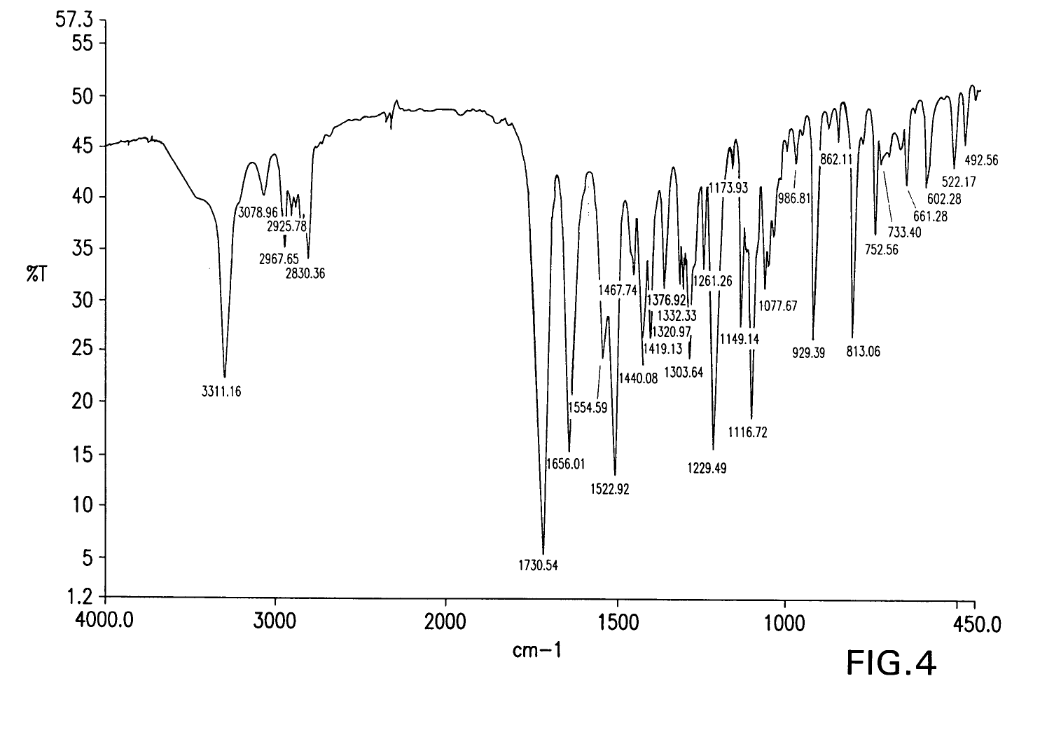

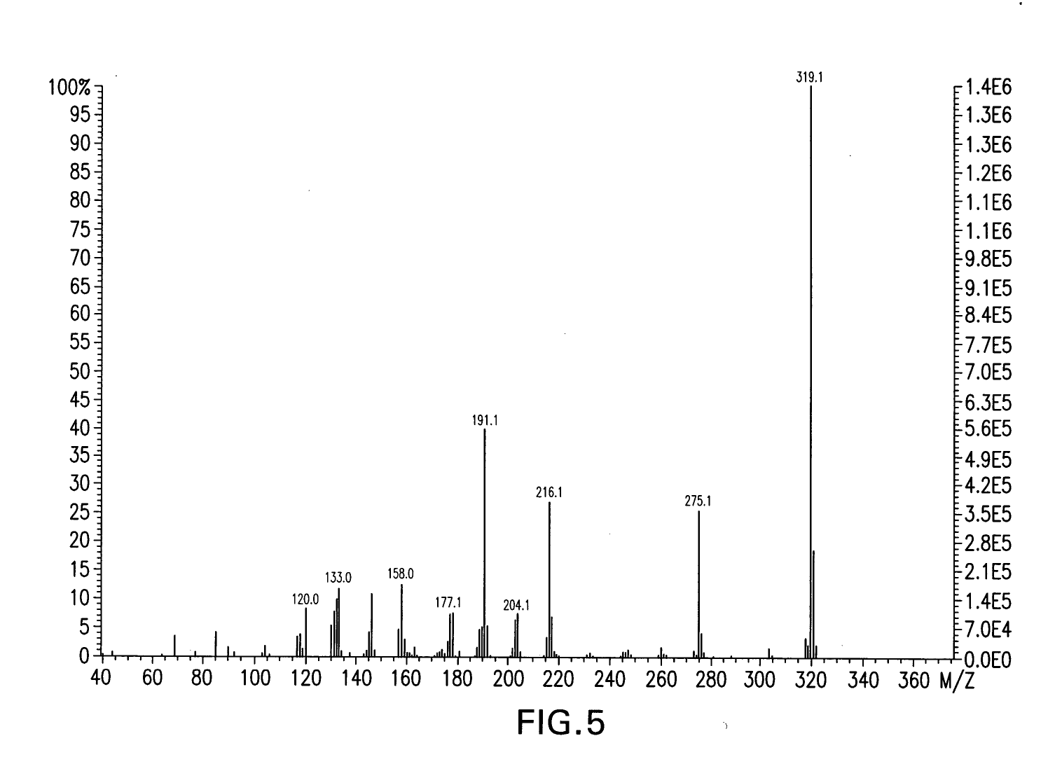

The isolated desfluoro linezolid of the present invention may be characterized by a ¹H NMR, substantially as depicted in FIG. 2. The isolated desfluoro linezolid of the present invention may be characterized by ¹³C NMR, substantially as depicted in FIG. 3. The isolated desfluoro linezolid of the present invention may be characterized by an IR spectrum substantially as depicted in FIG. 4. The isolated desfluoro linezolid of the present invention may be characterized by an Mass spectrum substantially as depicted in FIG. 5.

The isolated desfluoro linezolid of the present invention may be prepared by performing the process described in U.S. Pat. No. 5,688,792, with 1-fluoro-4-nitrobenzene instead of 3,4-difluoronitrobenzene, according to the following scheme:

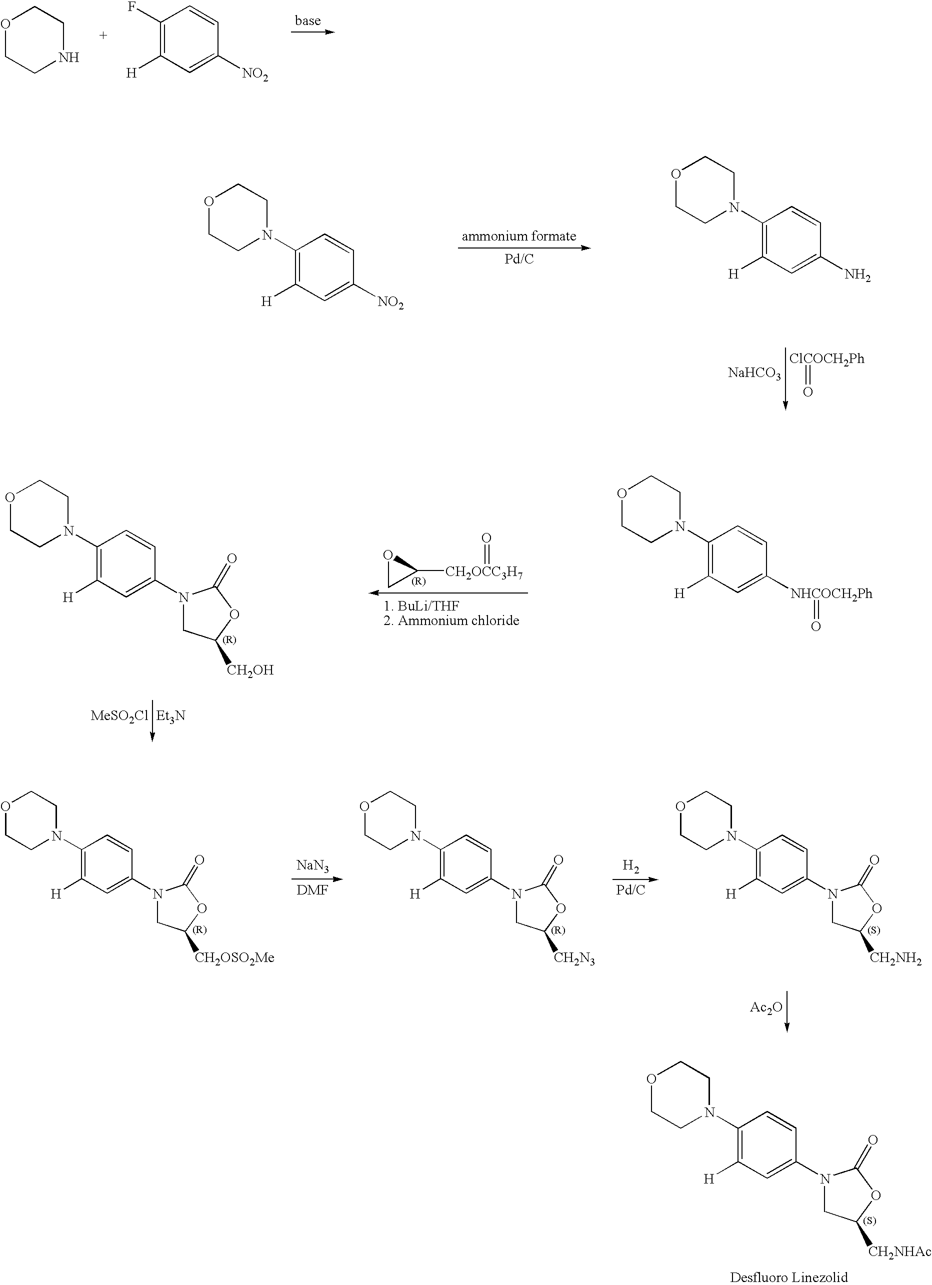

The desfluoro linezolid of the present invention is isolated by a process comprising the following steps; a) combining (5R)-[[3-[4-(4-morpholinyl)phenyl]-2-oxo-5-oxazolidinyl]methyl]azide with an organic solvent, preferably a C₁-C₄ alkyl ester or a C₆ to C₁₂ aromatic hydrocarbon, more preferably toluene or ethylacetate, most preferably toluene, and hydrogen gas in the presence of a catalyst to obtain a reaction mixture containing (5S)-[[3-[4-(4-morpholinyl)phenyl]-2-oxo-5-oxazolidinyl]methyl]amine; b) filtering the reaction mixture to obtain a solution containing (5S)-[[3-[4-(4-morpholinyl)phenyl]-2-oxo-5-oxazolidinyl]methyl]amine; c) adding acetic anhydride to the solution to obtain a precipitate; and d) recovering and drying the precipitate to obtain isolated desfluoro linezolid. Preferably, recovering of the precipitate in step d) is carried out by filtering or decanting. Preferably, the catalyst in step a) is selected from the group consisting of Pd/C, Raney Nickel, and noble metal catalysts, more preferably the catalyst is Pd/C.

The isolated desfluoro linezolid of the present invention is useful as a reference marker for linezolid. As such, it may be used in order to detect the desfluoro linezolid impurity in a linezolid sample.

For example, chromatography can be carried out on a reference sample and a linezolid sample. The resulting peaks can be compared to determine the presence of desfluoro linezolid. If the desfluoro linezolid is present in the linezolid sample, its relative location to linezolid allows for determining the RRT for the impurity as well as other impurities in the linezolid sample.

In one embodiment the present invention provides a method for detecting desfluoro linezolid impurity in a linezolid sample comprising:
a) providing a reference sample comprising desfluoro linezolid and linezolid;
b) carrying out chromatography, preferably HPLC, on the reference sample to determine the relative retention time of desfluoro linezolid compared to linezolid;
c) carrying out chromatography, preferably HPLC, on the linezolid sample to determine the relative retention time of an impurity compared to linezolid;
d) comparing the relative retention times determined in steps b) and c);
   wherein, if the relative retention times determined in steps b) and c) are substantially the same, the impurity is identified as desfluoro linezolid, being the same as in the reference sample.

The isolated desfluoro linezolid of the present invention is useful as a reference standard for linezolid, in order to quantify impurities in a linezolid sample. The present invention provides a method of determining the amount of the desfluoro linezolid impurity in a linezolid sample with chromatography, preferably HPLC, can comprise the following steps:
a) measuring by chromatography, preferably HPLC, the area under a peak in a chromatogram corresponding to desfluoro linezolid in a sample containing a known amount of desfluoro linezolid;
b) measuring by chromatography, preferably HPLC, the area under a peak in a chromatogram corresponding to desfluoro linezolid in a linezolid sample containing desfluoro linezolid; and
c) determining the amount of desfluoro linezolid in the linezolid sample by comparing the area of step a) to the area of step b).

The present invention provides an HPLC method for analyzing a sample containing at least one of linezolid and desfluoro linezolid comprising:
a) combining the sample with H₂O:ACN (3:1), preferably at a ratio of about 1:2.5 mg/ml, to obtain a solution;
b) injecting the solution of step (a) onto a silica column; and
c) eluting the sample from the column over a time period in the range from about 3 times to about 5 times the elution time of linezolid using a mixture A of K₂HPO₄ 0.01M:MeOH (80:20) and a mixture B of K₂HPO₄ 0.01M:MeOH (50:50) as an eluent; and
d) detecting at least one of linezolid and desfluoro linezolid in the relevant sample with a UV detector.
   Detection can be carried out at a wavelength of 254 nm. Detecting can include measuring at least one of the linezolid content and the desfluoro linezolid content. The elution time can be about 30 min to about 45 min, more preferably about 35 min. Preferably, eluting the sample in step c) is by a gradient which at time t=0 is 100% mixture A, at time t=15 min is a mixture of 57% mixture A and 43% mixture B, and at time t=25 min is a mixture of 35% mixture A and 65% mixture B. This HPLC method can be used as the HPLC chromatography in any of the methods of the present invention for detecting or determining the amount of the impurity desfluoro linezolid in a linezolid sample.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

| Sample | H₂O:ACN (3:1) at a ratio of about 1:2.5mg/ml, compared | | |
|---|---|---|---|
| preparation: | to the linezolid | | |
| Column: | Hypersil Gold 150 × 4.6, 5μ | | |
| Eluents: | K₂HPO₄ 0.01M:MeOH | A: 80:20 | B: 50:50 |
| Detection limit: | 0.1% | | |
| Detection at: | 254 nm | | |

TABLE 1
Elution Gradient

| Time | A | B | Flow |
|---|---|---|---|
| 0 | 100 | 0 | 1.5 |
| 15 | 57 | 43 | 2 |
| 25 | 35 | 65 | 2 |

Step 1. Preparation of N-(4-nitrophenyl)morpholine.

A solution of 1-fluoro-4-nitrobenzene (32 ml, 0.3 mol, Aldrich) in 50 ml of acetonitrile was added dropwise to a solution of morpholine (26 ml, 0.3 mol, Aldrich) and di-isopropylethylamine (51 ml, 0.33 mol, Merck) in 130 ml of acetonitrile with stirring. The reaction mixture was heated at reflux with stirring for 5 hours. The mixture was cooled to ambient temperature and kept overnight. The solid was filtered, washed with acetonitrile and gave after drying (40° C., vacuum 10 mm, 2 hour) N-(4-nitrophenyl)morpholine (46.5 g).

Step 2. Preparation of 4-(4-morpholinyl)aniline.

N-(4-nitrophenyl)morpholine (18.7 g) and (400 ml) ammonium formate (23.5 g) were suspended in a mixture tetrahydrofuran-methanol: 1 vol-4 vol (400 ml) and 10% Pd/C (0.5 g) was added to the stirred suspension by portion for 5 min. The mixture was stirred at ambient temperature for 3 hours. Then a mixture tetrahydrofuran-methanol: 1 vol-4 vol (100 ml) and ammonium formate (12 g) were added to the reaction mixture at once, followed by addition in portions of 10% Pd/C (0.5 g) with stirring. The reaction mixture was stirred for additional 4 hours. The solid was filtered, and the solvents from the mother liquor were evaporated under vacuum. The rest was triturated in a mixture ethyl acetate-water (80 ml-100 ml). The solid was filtered, dried (30° C., vacuum 10 mm, 8 hour) and gave 4-(4-morpholinyl)aniline (11.5 g).

Step 3. Preparation of 4-(4-morpholinyl)-N-benzyloxycarbonylaniline.

A solution of benzylchloroformate (9.2 ml, 65 mmol, Aldrich) in 10 ml of acetone was added dropwise to a stirred suspension of 4-(4-morpholinyl)-N-benzyloxycarbonylaniline (11.5 g, 65 mmol) and sodium bicarbonate (11 g, 130 mmol) in a mixture acetone water (300 ml-150 ml) at 0-5° C. for a half hour. Then the mixture was stirred at ambient temperature for 5 hours. The solid was filtered, and the solvents from the mother liquor were evaporated under vacuum. The rest was dissolved in dichloromethane-water (300 ml-100 ml). After phase separation dichloromethane was evaporated under vacuum and gave 4-(4-morpholinyl)-N-benzyloxycarbonylaniline (17 g)

Step 4. Preparation of (5R)-3-[[(4-morpholinyl)phenyl]-2-oxo-5-oxazolidinyl]methanol.

A solution of BuLi in hexane (1.6M, 43 ml, 68 mmol) was added dropwise to a solution of 4-(4-morpholinyl)-N-benzyloxycarbonylaniline (16 g, 51 mmol) in 300 ml of tetrahydrofuran at −70° C. during an hour in nitrogen atmosphere. Then a solution of R-glycidyl butyrate (8 g, 5 mmol, Aldrich) in 20 ml of tetrahydrofuran was added to the stirred reaction mixture at the same temperature for a half hour. The reaction mixture was stirred at −60 to −70° C. for additional 6 hours. The reaction mixture was kept at the ambient temperature for 3 days. Subsequently, saturated aqueous ammonium chloride (10 ml) and then water (200 ml) were added to the mixture and stirred for 2 hours. The organic phase was separated; and the water phase was extracted with ethyl acetate (2×90 ml). The combined organic phase was washed with brine, the solvents were evaporated under vacuum. The rest was crystallized from methanol and gave (5R)-[[3-(4-morpholinyl)phenyl]-2-oxo-5-oxazolidinyl]methanol (8 g).

Step 5. Preparation of (5R)-[[3-(4-morpholinyl)phenyl]-2-oxo-5-oxazolidinyl]methanesulfonate.

(5R)-[[3-(4-Morpholinyl)phenyl]-2-oxo-5-oxazolidinyl]methanol (8.4 g) was suspended in 70 ml of dichloromethane, contained triethylamine (9.5 ml). Methanesulfonylchloride (5.2 ml) was added to the cooled stirred suspension for 20 min at 0° C. Then the mixture was stirred at room temperature for 4 hours. The reaction mixture was poured in stirred mixture water-ethyl acetate (70 ml-15 ml) and stirred at room temperature for additional 30 min. The precipitate was filtered, washed with water and dichloromethane and gave after drying under vacuum (5R)-[[3-(4-morpholinyl)phenyl]-2-oxo-5-oxazolidinyl]methanesulfonate (8.5 g).

Step 6. Preparation of (5R)-[[3-(4-morpholinyl)phenyl]-2-oxo-5-oxazolidinyl]methyl]azide.

(5R)-[[3-(4-morpholinyl)phenyl]-2-oxo-5-oxazolidinyl]methanesulfonate (8.4 g) was suspended in 60 ml of dimethylformamide. Sodium azide (2.3 g) was added to the suspension and the mixture was heated at 80° C. with stirring for 1.5 hours, and then at room temperature overnight. The reaction mixture was poured in 200 ml of water and stirred for 2.5 hours. After filtration, washing and drying (30° C., vacuum 10 mm, overnight) (5R)-[[3-(4-morpholinyl)phenyl]-2-oxo-5-oxazolidinyl]methyl]azide (6.5 g) was obtained.

Step 7. Preparation of N-[[(5S)-3-[4-(4-morpholinyl)phenyl]-2-oxo-5-oxazolidinyl]methyl]acetamide (des-fluoro-linezolid).

In a 1 L reactor, 6 g (5R)-[[3-(4-morpholinyl)phenyl]-2-oxo-5-oxazolidinyl]methyl]azide were charged with 0.7 L toluene followed by 0.6 g Pd/C (10% Pd/C containing 52% water). The system was bubbled with ammonia (gas) during 2 h, and then flushed three times with nitrogen and 3 times with hydrogen. The pressure of hydrogen was set to 1.5 atm. The reaction mixture was stirred at RT and the reaction followed up until completion. The reaction mixture was filtered and the solution was treated with 60 ml acetic anhydride at RT. The precipitate was filtered and dried to obtain 3.3 g of desfluoro linezolid (purity: 99.3%).

Desfluoro Linezolid ¹H-NMR and ¹³C-NMR Identification

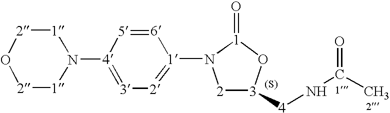

| | C | H | | |
|---|---|---|---|---|
| N^{o} | Chem. Shift, ppm | Chem. Shift, ppm | Multiplicity | J, Hz |
| 1 | 154.7 | ˜ | ˜ | ˜ |
| 2a | 48.0 | 4.04 | t | 8.8 |
| b | | 3.68 | m | ˜ |
| 3 | 71.7 | 4.67 | m | ˜ |
| 4 | 41.9 | 3.40 | t | 5.3 |
| 1′ | 130.9 | ˜ | ˜ | ˜ |
| 2′ | 119.9 | 7.37 | d | 8.8 |
| 3′ | 115.9 | 6.95 | d | 8.8 |
| 4′ | 148.0 | ˜ | ˜ | ˜ |
| 5′ | 115.9 | 6.95 | d | 8.8 |
| 6′ | 119.9 | 7.37 | d | 8.8 |
| 1″ | 49.2 | 3.04 | brt | 4.4 |
| 2″ | 66.5 | 3.72 | brt | 4.6 |
| 1′′′ | 170.0 | ˜ | ˜ | ˜ |
| 2′′′ | 22.8 | 1.83 | s | ˜ |
| NH | ˜ | 8.21 | t | 5.5 |

What is claimed is:

1. Isolated desfluoro linezolid.

2. The isolated desfluoro linezolid of claim 1, characterized by data selected from: ¹H NMR (400 MHz, DMSO-d6) δ (ppm): 1.83 (s), 3.04 (brt), 3.40 (t), 3.68 (m), 3.72 (brt), 4.04 (t), 4.67 (m), 6.95 (d), 6.95 (d), 7.37 (d), 7.37 (d) and 8.21 (t); ¹³C NMR (100 MHz, DMSO-d6) δ (ppm): 22.8, 41.9, 48.0, 49.2, 66.5, 71.7, 115.9, 115.9, 119.9, 119.9, 130.9, 148.0, 154.7, 170.0; EI+m/z (MH⁺): 319; and IR spectra on KBr at 1523, 1555, 1656, 1731, 2830, 2926, 2968 and 3311 cm⁻¹.

3. The isolated desfluoro linezolid of claim 1, containing less than about 5% by weight of linezolid.

4. The isolated desfluoro linezolid of claim 3, containing less than about 2% by weight of linezolid.

5. The isolated desfluoro linezolid of claim 4, containing less than about 1% by weight of linezolid.

6. A method of preparing the isolated desfluoro linezolid of claim 1 comprising:
a) combining (5R)-[[3-[4-(4-morpholinyl)phenyl]-2-oxo-5-oxazolidinyl]methyl]azide with an organic solvent and hydrogen gas in the presence of a catalyst to obtain a reaction mixture containing (5S)-[[3-[4-(4-morpholinyl)phenyl]-2-oxo-5-oxazolidinyl]methyl]amine;
b) filtering the reaction mixture to obtain a solution containing (5S)-[[3-[4-(4-morpholinyl)phenyl]-2-oxo-5-oxazolidinyl]methyl]amine;
c) adding acetic anhydride to the solution to obtain a precipitate; and
d) recovering and drying the precipitate to obtain isolated desfluoro linezolid.

7. The method of claim 6, wherein the organic solvent is a C₁-C₄ alkyl ester or a C₆-C₁₂ aromatic hydrocarbon.

8. The method of claim 7, wherein the organic solvent is toluene.

9. The method of claim 6, wherein the catalyst in step a) is Pd/C.

10. A method of detecting a desfluoro linezolid impurity in a linezolid sample comprising:
a) providing a reference sample comprising desfluoro linezolid and linezolid;
b) carrying out HPLC chromatography on the reference sample to determine the relative retention time of desfluoro linezolid compared to linezolid;
c) carrying out HPLC chromatography on the linezolid sample to determine the relative retention time of an impurity compared to linezolid;
d) comparing the relative retention times determined in steps b) and c);
wherein, if the relative retention times determined in steps b) and c) are substantially the same, the impurity is identified as desfluoro linezolid.

11. A method of determining the amount of a desfluoro linezolid impurity in a linezolid sample comprising:
a) measuring by HPLC chromatography the area under a peak in an HPLC chromatogram corresponding to desfluoro linezolid in a sample comprising a known amount of desfluoro linezolid;
b) measuring by HPLC chromatography the area under a peak in an HPLC chromatogram corresponding to desfluoro linezolid in a linezolid sample containing desfluoro linezolid; and
c) determining the amount of desfluoro linezolid in the linezolid sample by comparing the area of step a) to the area of step b).

12. The method of claim 11, wherein the sample in step a) is reference standard.

13. An HPLC method for analyzing a sample containing at least one of linezolid and desfluoro linezolid comprising:
a) combining the sample with H₂O:ACN (3:1) to obtain a solution;
b) injecting the solution of step (a) into a silica column; and
c) eluting the sample from the column over a time period in the range from about 3 times to about 5 times the elution time of linezolid using a mixture A of K₂HPO₄ 0.01M:MeOH (80:20) and a mixture B of K₂HPO₄ 0.01M:MeOH (50:50) as an eluent; and
d) detecting at least one of linezolid and desfluoro linezolid in the relevant sample with a UV detector.

14. The method of claim 13, wherein combining the sample with H2O:ACN (3:1) is at a ratio of 1:2.5 mg/ml.

15. The method of claim 13, wherein detecting at least one of linezolid and desfluoro linezolid in step d) comprises measuring at least one of the linezolid content and the desfluoro linezolid content.

16. The method of claim 13, wherein the time period in step c) is about 30 min to about 45 min.

17. The method of claim 16, wherein the time period in step c) is about 35 min.

18. The method of claim 13, wherein eluting the sample in step c) is by a gradient which at time t=0 is 100% mixture A, at time t=15 min is a mixture of 57% mixture A and 43% mixture B, and at time t=25 min is a mixture of 35% mixture A and 65% mixture B.
